(19) United States
(12) Patent Application Publication
(10) Pub. No.: US 2009/0118249 A1
(43) Pub. Date: May 7, 2009

(54) DHEAS INHALATION COMPOSITIONS

(76) Inventors: Ingo Friedrich, Aachen (DE); Manfred Keller, Munich (DE); Stefan Kerscher, Munich (DE)

Correspondence Address: WILSON SONSINI GOODRICH & ROSATI, 650 PAGE MILL ROAD, PALO ALTO, CA, 94304-1050, US

(21) Appl. No.: 12/204,693

(22) Filed: Sep. 4, 2008

Related U.S. Application Data
(60) Provisional application No. 60/970,869, filed on Sep. 7, 2007.

(51) Int. Cl. A61K 31/56; A61P 11/00

(52) U.S. Cl. 514/178

(57) ABSTRACT

The present invention provides compositions for aqueous suspension comprising DHEAS and a divalent cation. The suspension in combination with a nebulizer or nasal pump spray can be administers as an aerosol for the treatment of respiratory diseases and conditions. The present invention also provides methods for making compositions in form of aqueous suspension of DHEA and divalent cations.

### CROSS REFERENCE

This application claims the benefit of U.S. Provisional Application No. 60/970,869, filed Sep. 7, 2007, which application is incorporated herein by reference in its entirety.

### FIELD OF THE INVENTION

This invention relates to compositions for inhalation that are useful for aerosol administration for the treatment of respiratory diseases and conditions. The invention also relates to methods of making compositions for inhalation. The compositions for inhalation are based on compositions comprising dehydroepiandrosterone sulfate (DHEAS) in a form for respiratory administration with, for example, a nebulizer, or an atomizer.

### BACKGROUND OF THE INVENTION

Respiratory disease and conditions, such as COPD, asthma, allergic rhinitis, Acute Respiratory Distress Syndrome (ARDS), pulmonary fibrosis, cystic fibrosis, and cancers of the respiratory system are common diseases in industrialized countries, and in the United States alone account for extremely high health care costs. These diseases or conditions have recently been increasing at an alarming rate, both in terms of prevalence, morbidity and mortality. In spite of this, their underlying causes still remain poorly understood.

Chronic obstructive pulmonary disease (COPD) causes a continuing obstruction of airflow in the airways. COPD is characterized by airflow obstruction that is generally caused by chronic bronchitis, emphysema, or both. Commonly, the airway obstruction is mostly irreversible. In chronic bronchitis, airway obstruction results from chronic and excessive secretion of abnormal airway mucus, inflammation, bronchospasm, and infection. Chronic bronchitis is also characterized by chronic cough, mucus production, or both, for at least three months in at least two successive years where other causes of chronic cough have been excluded. In emphysema, a structural element (elastin) in the terminal bronchioles is destroyed leading to the collapse of the airway walls and inability to exhale “stale” air. In emphysema there is permanent destruction of the alveoli. Emphysema is characterized by abnormal permanent enlargement of the air spaces distal to the terminal bronchioles, accompanied by destruction of their walls and without obvious fibrosis. COPD can also give rise to secondary pulmonary hypertension. Secondary pulmonary hypertension itself is a disorder in which blood pressure in the pulmonary arteries is abnormally high. In severe cases, the right side of the heart must work harder than usual to pump blood against the high pressure. If this continues for a long period, the right heart enlarges and functions poorly, and fluid collects in the ankles (edema) and belly.

COPD characteristically affects middle aged and elderly people, and is one of the leading causes of morbidity and mortality worldwide. In the United States it affects about 14 million people and is the fourth leading cause of death, and the third leading cause for disability in the United States. Both morbidity and mortality, however, are rising. The estimated prevalence of this disease in the United States has risen by 41% since 1982, and age adjusted death rates rose by 71% between 1966 and 1985. This contrasts with the decline over the same period in age-adjusted mortality from all causes (which fell by 22%), and from cardiovascular diseases (which fell by 45%). In 1998 COPD accounted for 112,584 deaths in the United States.

Asthma is a condition characterized by variable, in many instances reversible obstruction of the airways. This process is associated with lung inflammation and in some cases lung allergies. Many patients have acute episodes referred to as “asthma attacks,” while others are afflicted with a chronic condition. The asthmatic process is believed to be triggered in some cases by inhalation of antigens by hypersensitive subjects. This condition is generally referred to as “extrinsic asthma.” Other asthmatics have an intrinsic predisposition to the condition, which is thus referred to as “intrinsic asthma,” and may be comprised of conditions of different origin, including those mediated by the adenosine receptor(s), allergic conditions mediated by an immune IgE-mediated response, and others. Many asthma sufferers have a group of symptoms, which are characteristic of this condition: bronchoconstriction, lung inflammation and decreased lung surfactant. Existing bronchodilators and anti-inflammatories are currently commercially available and are prescribed for the treatment of asthma. The most common anti-inflammatories, corticosteroids, have considerable side effects but are commonly prescribed nevertheless. Most of the drugs available for the treatment of asthma are, more importantly, barely effective in a small number of patients.

Acute Respiratory Distress Syndrome (ARDS) is also known in the medical literature as stiff lung, shock lung, pump lung and congestive atelectasis, and its incidence is 1 out of 100,000 people. ARDS is believed to be caused by a failure of the respiratory system characterized by fluid accumulation within the lung that, in turn, causes the lung to stiffen. The condition is triggered by a variety of processes that injure the lungs. In general, ARDS occurs as a medical emergency. It may be caused by a variety of conditions that directly or indirectly cause the blood vessels to “leak” fluid into the lungs. In ARDS, the ability of the lungs to expand is severely decreased and damage to the air sacs and lining (endothelium) of the lung is extensive. The concentration of oxygen in the blood remains very low in spite of high concentration of supplemental oxygen that is generally administered to a patient. Among the systemic causes of lung injury are trauma, head injury, shock, sepsis, multiple blood transfusions and medications. Pulmonary causes include pulmonary embolism, severe pneumonia, smoke inhalation, radiation, high altitude, near drowning, and others like cigarette smoking. ARDS symptoms usually develop within 24 to 48 hours of the occurrence of an injury or illness.

ARDS' most common symptoms are labored, rapid breathing, nasal flaring, cyanosis blue skin, lips and nails caused by lack of oxygen to the tissues, breathing difficulty, anxiety, stress, tension, joint stiffness, pain and temporarily absent breathing. ARDS is commonly diagnosed by testing for symptomatic signs, for example by a simple chest auscultation or examination with a stethoscope that may reveal abnormal symptomatic breath sounds. In some cases ARDS appears to be associated with other diseases, such as acute myelogenous leukemia, with acute tumor lysis syndrome (ATLS) developed after treatment with, e.g. cytosine arabinoside. In general, however, ARDS appears to be associated with traumatic injury, severe blood infections such as sepsis, or other systemic illness, high dose radiation therapy and chemotherapy, and inflammatory responses which lead to multiple organ failure, and in many cases death. In premature babies (“premies”), the lungs are not quite developed and, therefore, the fetus is in an anoxic state during development. When premies survive RDS, they frequently develop bronchopulmonary dysplasia (BPD), also called chronic lung disease of early infancy, which is often fatal.

Rhinitis may be seasonal or perennial, allergic or non-allergic. Non-allergic rhinitis may be induced by infections, such as viruses, or associated with nasal polyps, as occurs in patients with aspirin idiosyncrasy. Medical conditions such as pregnancy or hypothyroidism and exposure to occupational factors or medications may cause rhinitis. Allergic rhinitis afflicts one in five Americans, accounting for an estimated $4 to 10 billion in health care costs each year, and occurs at all ages. Because many people mislabel their symptoms as persistent colds or sinus problems, allergic rhinitis is probably underdiagnosed. Typically, IgE combines with allergens in the nose to produce release of chemical mediators, induction of cellular processes, and neurogenic stimulation, causing an underlying inflammation. Symptoms include nasal congestion, discharge, sneezing, and itching, as well as itchy, watery, swollen eyes. Over time, allergic rhinitis sufferers often develop sinusitis, otitis media with effusion, and nasal polyposis, and may exacerbate asthma, and is associated with mood and cognitive disturbances, fatigue and irritability.

Pulmonary fibrosis, interstitial lung disease (ILD), or interstitial pulmonary fibrosis, include more than 130 chronic lung disorders that affect the lung by damaging lung tissue, and producing inflammation in the walls of the air sacs in the lung, scarring or fibrosis in the interstitium (or tissue between the air sacs), and stiffening of the lung, thus the name of the disease. Although the progress and symptoms of pulmonary fibrosis and other ILDs may vary from person to person, they have one common link: they affect parts of the lung. When inflammation involves the walls of the bronchioles (small airways), it is called bronchiolitis, when it involves the walls and air spaces of the alveoli (air sacs), it is called alveolitis, and when it involves the small blood vessels (capillaries) of the lungs, it is called vasculitis. The inflammation may heal, or it may lead to permanent scarring of the lung tissue, in which case it is called pulmonary fibrosis. This fibrosis or scarring of the lung tissue results in permanent loss of its ability to breathe and carry oxygen, and the amount of scarring determines the level of disability a person experiences because of the destruction by the scar tissue of the air sacs and lung tissue between and surrounding the air sacs and the lung capillaries. Many of the diseases are often named after the occupations with which they are associated, such as Grain handlier's lung, Mushroom worker's lung, Bagassosis, Detergent worker's lung, Maple bark stripper's lung, Malt worker's lung, Paprika splitter's lung, and Bird breeder's lung. “Idiopathic” (of unknown origin) pulmonary fibrosis (IPF) is the label applied when all other causes of interstitial lung disease have been ruled out, and is said to be caused by viral illness and allergic or environmental exposure (including tobacco smoke). Bacteria and other microorganisms are not thought to be a cause of IPF. There is also a familial form of the disease, known as familial idiopathic pulmonary fibrosis whose main symptom is shortness of breath. Since many lung diseases show this symptom, making a correct diagnosis is often difficult. The shortness of breath may first appear during exercise and the condition may progress then to the point where any exertion is impossible. Eventually resulting in shortness of breath even at rest. Other symptoms may include a dry cough (without sputum), and clubbing of the fingertips.

Cancer is one of the most prevalent and feared diseases of our times. It generally results from the carcinogenic transformation of normal cells of different epithelia. Two of the most damaging characteristics of carcinomas and other types of malignancies are their uncontrolled growth and their ability to create metastases in distant sites of the host, particularly a human host. Cancer can occur in any tissue making up the respiratory system, including all the organs involved in the breathing process such as the lungs, bronchi and throat. Lung cancer, oral cancer and throat cancer are some examples of respiratory system cancers. The treatment of cancer presently relies on surgery, irradiation therapy and systemic therapies such as chemotherapy, different immunity-boosting medicines and procedures, hyperthermia and systemic, radioactively labeled monoclonal antibody treatment, immunotoxins and chemotherapeutic drugs. Cancer of the respiratory system can be treated by drugs delivered as an inhalant.

Dehydroepiandrosterone (DHEA) is a naturally occurring steroid secreted by the adrenal cortex with apparent chemoprotective properties. Epidemiological studies have shown that low endogenous levels of DHEA correlate with increased risk of developing some forms of cancer, such as pre-menopausal breast cancer in women and bladder cancer in both sexes. The ability of DHEA and DHEA analogues, e.g. dehydroepiandrosterone sulfate (DHEAS), to inhibit carcinogenesis is not clear but one suggestion is that it results from their non-competitive inhibition of the activity of the enzyme glucose 6-phosphate dehydrogenase (G6PDH). G6PDH is the rate limiting enzyme of the hexose monophosphate pathway, a major source of intracellular ribose-5-phosphate and NADPH. Ribose-5-phosphate is a necessary substrate for the synthesis of both ribo- and deoxyribonucleotides required for the synthesis of RNA and DNA. NADPH is a cofactor also involved in nucleic acid biosynthesis and the synthesis of hydroxymethylglutaryl Coenzyme A reductase (HMG CoA reductase). HMG CoA reductase is an unusual enzyme that requires two moles of NADPH for each mole of product, mevalonate, produced. Thus, it appears that HMG CoA reductase would be ultrasensitive to DHEA-mediated NADPH depletion, and that DHEA-treated cells would rapidly show the depletion of intracellular pools of mevalonate. Mevalonate is required for DNA synthesis, and DHEA arrests human cells in the G1 phase of the cell cycle in a manner closely resembling that of the direct HMG CoA. Because G6PDH produces mevalonic acid used in cellular processes such as protein isoprenylation and the synthesis of dolichol, a precursor for glycoprotein biosynthesis, DHEA inhibits carcinogenesis by depleting mevalonic acid and thereby inhibiting protein isoprenylation and glycoprotein synthesis. Mevalonate is a central precursor for the synthesis of cholesterol, as well as for the synthesis of a variety of non-sterol compounds involved in post-translational modification of proteins, such as farnesyl pyrophosphate and geranyl pyrophosphate. Mevalonate is also a central precursor for the synthesis of dolichol, a compound that is required for the synthesis of glycoproteins involved in cell-to-cell communication and cell structure. Mevalonate is also central to the manufacture of ubiquinone, an anti-oxidant with an established role in cellular respiration. It has long been known that patients receiving steroid hormones of adrenocortical origin at pharmacologically appropriate doses show increased incidence of infectious disease.

DHEA, also known as (3.beta.)-3-hydroxyandrost-5-en-17-one, or dehydroisoandrosterone, is a 17-ketosteroid which is quantitatively one of the major adrenocortical steroid hormones found in mammals. Although DHEA appears to serve as an intermediary in gonadal steroid synthesis, the primary physiological function of DHEA has not been fully understood. It has been known, however, that levels of this hormone begin to decline in the second decade of life, reaching 5% of the original level in the elderly. Clinically, DHEA has been used systemically and/or topically for treating patients suffering from psoriasis, gout, hyperlipemia, and it has been administered to post-coronary patients. In mammals, DHEA has been shown to have weight optimizing and anti-carcinogenic effects, and it has been used clinically in Europe in conjunction with estrogen as an agent to reverse menopausal symptoms and also has been used in the treatment of manic depression, schizophrenia, and Alzheimer's disease. DHEA has also been used clinically at 40 mg/kg/day in the treatment of advanced cancer and multiple sclerosis. Mild androgenic effects, hirsutism, and increased libido are the side effects observed. These side effects can be overcome by monitoring the dose and/or by using analogues. The subcutaneous or oral administration of DHEA to improve the host's response to infections is known, as is the use of a patch to deliver DHEA. DHEA is also known as a precursor in a metabolic pathway that ultimately leads to more powerful agents that increase immune response in mammals. That is, DHEA acts as a biphasic compound: it acts as an immuno-modulator when converted to androstenediol or androst-5-ene-3.beta., 17.beta.-diol (.beta.AED), or androstenetriol or androst-5-ene-3.beta., 7.beta., 17.beta.-triol (.beta.AET). However, in vitro DHEA has certain lymphotoxic and suppressive effects on cell proliferation prior to its conversion to PAED and/or PAET. It is, therefore, believed that the superior immunity enhancing properties obtained by administration of DHEA result from its conversion to more active metabolites.

U.S. Pat. No. 5,660,835 (and corresponding PCT publication WO 96/25935) discloses a novel method of treating asthma or adenosine depletion in a subject by administering to the subject a dehydroepiandrosterone (DHEA) or DHEA-related compound. The patent also discloses a novel pharmaceutical composition in regards to an inhalable or respirable formulation comprising DHEA or DHEA-related compounds that is in a respirable particle size.

U.S. Pat. No. 5,527,789 discloses a method of combating cancer in a subject by administering to the subject a DHEA or DHEA-related compound, and ubiquinone to combat heart failure induced by the DHEA or DHEA-related compound. U.S. Pat. No. 6,087,351 discloses an in vivo method of reducing or depleting adenosine in a subject's tissue by administering to the subject a DHEA or DHEA-related compound. U.S. Pat. No. 5,859,000 discloses methods for reducing mast cell mediated allergic reactions including mast cell mediated allergy and asthma by administering a DHEA derivative. U.S. patent application Ser. No. 10/454,061, filed Jun. 3, 2003, discloses a method for treating COPD in a subject by administering to the subject a DHEA or DHEA-related compound. U.S. patent application Ser. No. 10/462,901, filed Jun. 17, 2003, discloses a stable dry powder formulation of DHEA in an aerosolizable form sealed in a container. U.S. patent application Ser. No. 10/462,927, filed Jun. 17, 2003, discloses a stable dry powder formulation of dihydrate crystal form of DHEAS suitable for treating asthma and COPD.

The aerosol dosage form provides an effective means of delivering drugs into the respiratory system. Aerosols can be delivered directly to the airways, for instance, by metered dose inhalers, nebulizers, or dry powder inhaler. The aerosol form is a desirable method of delivering DHEA or DHEAS to the upper and lower respiratory system of a patient. There is a need for inhalation formulations of DHEA that can be delivered in aerosol form either as aqueous or non aqueous systems to the lower and/or upper respiratory tract.

### SUMMARY OF THE INVENTION

The present invention provides compositions for administering DHEAS in an aqueous nebulizable aerosol form and methods of making such compositions.

In one aspect of the present invention is a composition for inhalation via a nebulizer comprising a divalent cation creating an aqeuous suspension of DHEAS. In some embodiments, the divalent ion comprises an alkaline earth metal. In some embodiments, the divalent ion comprises magnesium. In another aspect, the invention provides a composition for inhalation comprising a salt of DHEAS wherein the counterion to DHEAS comprises a divalent cation.

In some embodiments, the molar ratio of divalent cation to DHEAS in the composition is between about 0.5 and 5. In some embodiments, the molar ratio of divalent cation to DHEAS is between about 0.25 and 4. In some embodiments, the molar ratio of divalent cation to DHEAS is between about 0.75 to 1.25.

In some embodiments, the amount of DHEAS in the suspension is between about 0.5 wt. % and 10 wt. %. In some embodiments, the amount of DHEAS in the suspension is between about 1 wt. % and 10 wt. %. In some embodiments, the amount of DHEAS in the suspension is between about 2 wt. % and 5 wt. %. In some embodiments, the amount of DHEAS in the suspension is about 3.5 wt. %.

The compositions can further comprise an excipient, and in some embodiments, the excipient can comprise a sugar or sugar alcohol. In some embodiments, the excipient comprises xylitol, mannitol, trehalose, fructose, sucrose which can stabilize the formulation and act due to their sweet taste as taste modifying agents, as well.

The compositions can further comprise a sweetener that are not derived from sugars or sugar alcohols, and the sweetening agent can comprise saccharine, or its sodium salt, aspartame or other sweeteners approved for pharmaceutical products.

The compositions can further comprise a flavoring agent, and the flavoring agent can comprise levomenthol.

The compositions can further comprise a preservative, Suitable preservatives include but are not limited to C12 to C15 alkyl benzoates, and alkyl p-hydroxybenzoates (including methyl 4-hydroxybenzoate, ethyl 4-hydroxybenzoate, propyl 4-hydroxybenzoate, and suitable salts thereof). In some embodiments the preservative comprises propyl-4-hydroxybenzoate.

In some embodiments, the compositions further comprise an emulsifier or surfactant. In some embodiments, the emulsifier or surfactant is Vitamin E-TPGS. The emulsifier Vitamin E-TPGS can act as an oxygen or radical scavenger and can stabilize the formulation due to its antioxidant properties in addition to acting as an emulsifier.

In some embodiments an antioxidant or radical scavenger other than Vitamin E TPGS can be used. For example, other Vitamin E derivatives may be employed.

One aspect of the invention is a method of making a composition for inhalation comprising the steps of; mixing DHEAS in a first aqueous volume; mixing a compound comprising a divalent cation in a second aqueous volume: and combining the aqueous volumes to form a suspension of DHEAS.

Some embodiments further comprise the step of homogenizing the suspension of DHEAS.

In some embodiments, the divalent cation comprises an alkaline earth metal. In some embodiments, the divalent cation comprises magnesium in form of its water soluble salts, such as magnesium chloride, -sulfate, -gluconate, or -aspartate.

In some embodiments, the compound comprising the divalent cation is magnesium chloride.

Some embodiments further comprise mixing an excipient into the first aqueous volume, the second aqueous volume, or both the first and second aqueous volumes. In some embodiments, the excipient comprises a sugar alcohol, such as xylitol or mannitol or sugars, such as sucrose, trehalose or fructose.

Some embodiment further comprise mixing a sweetening agent into the first aqueous volume, the second aqueous volume or both the first and second aqueous volumes. In some embodiments, the sweetening agent comprises saccharine or saccharin-sodium.

Some embodiments further comprise mixing a flavoring agent into the first aqueous volume, the second aqueous volume or both the first and second aqueous volumes. In some embodiments, the flavoring agent comprises levomenthol

Some embodiments further comprise mixing a preservative into the first aqueous volume, the second aqueous volume or both the first and second aqueous volumes. In some embodiments, the preservative comprises a methyl, ethyl, or propyl-4-hydroxybenzoate.

Some embodiments further comprise mixing an emulsifier or surfactant into the first aqueous volume, the second aqueous volume or both the first and second aqueous volumes. In some embodiments, the emulsifier or surfactant is Vitamin E-TPGS.

In some embodiments, the first aqueous volume is acidic. In some embodiments the first aqueous volume is alkaline. In some embodiments, an aqueous buffer system is used for the adjustment of the pH to improve the physical and chemical stability of the formulation.

Some embodiments further comprise the addition of HCl to the first aqueous volume.

Some embodiments further comprise homogenizing the suspension formed by mixing the first and second aqueous volumes.

One aspect of the present invention is a method of making an composition for inhalation comprising the steps of; mixing DHEAS sodium salt, an excipient, a preservative, a sweetening agent, an emulsifier, and a flavoring agent in an first aqueous volume; mixing a compound comprising magnesium chloride in a second aqueous volume; combining the first and second aqueous volumes to form a suspension of DHEAS; and homogenizing the suspension.

In some embodiments, the excipient comprises xylitol or mannitol.

In some embodiments, the preservative comprises a methyl, ethyl, or propyl-4-hydroxybenzoate

In some embodiments, the sweetening agent is saccharine or saccharine-sodium

In some embodiments, the emulsifier is Vitamin E-TPGS.

In some embodiments, the flavoring agent is levomenthol.

In some embodiments, the combining of the first aqueous and second aqueous volume comprises adding the second aqueous volume to the first aqueous volume in a controlled manner.

One aspect of the invention is an aqueous suspension formed from the process comprising the steps of; mixing DHEAS in an first aqueous volume; mixing a compound comprising a divalent cation in a second aqueous volume: and mixing the aqueous volumes to form a suspension of DHEAS.

In some embodiments, the molar ratio of divalent cation to DHEAS is between about 0.5 and 5. In some embodiments, the molar ratio of divalent cation to DHEAS is between about 0.25 and 4. In some embodiments, the molar ratio of divalent cation to DHEAS is between about 0.75 to 1.25.

In some embodiments, the amount of DHEAS in the suspension is between about 0.5 wt. % and 10 wt. %. In some embodiments, the amount of DHEAS in the suspension is between about 1 wt. % and 10 wt. %. In some embodiments, the amount of DHEAS in the suspension is between about 2 wt. % and 5 wt. %. In some embodiments, the amount of DHEAS in the suspension is about 3.5 wt. %.

The aqueous suspension can further comprise an excipient, and in some embodiments, the excipient can comprise a sugar or a sugar alcohol. In some embodiments, the excipient comprises xylitol or mannitol.

The aqueous suspension can further comprise a sweetener, and the sweetening agent can comprise saccharine or saccharine sodium.

The aqueous suspension can further comprise a flavoring agent, and the flavoring agent can comprise levomenthol.

The aqueous suspension can further comprise a preservative, and the preservative can comprise methyl, ethyl, or propyl-4-hydroxybenzoate.

In some embodiment, the aqueous suspension can further comprise a buffer for the adjustment of the pH to improve the physical and chemical stability of the formulation.

In some embodiments, the aqueous suspension can further comprise an emulsifier such as Vitamin E-TPGS.

In some embodiments, the aqueous suspension comprises a pharmaceutically acceptable buffer for adjusting the pH of the aqueous suspension to between about 5 and about 8. In some embodiments, the pharmaceutically acceptable buffer is for adjusting the pH to between about 6 and about 7.5.

In some embodiments, the aqueous suspension has an osmolality between 200 and 500 mosmol/kg

One aspect of the invention is a method of treating an animal comprising; nebulizing the compositions of the invention with a nebulizer capable of an emitted dose of greater than 50% of the nominal dose wherein greater than 50% of the emitted composition comprises droplets less than or equal to about 5 μm in diameter. In some embodiments, the emitted dose is the dose emitted via mouthpiece or face mask. In some embodiments, the emitted composition has mass median aerodynamic diameter (MMAD) between about 2 and about 5 μm. In some embodiments, the emitted composition has mass median aerodynamic diameter (MMAD) between about 3 and about 4 μm. In some embodiments, the emitted composition has a geometric standard deviation (GSD) of less than about 2.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

The term “agent”, as used herein, means a chemical compound, a mixture of chemical compounds, a synthesized compound, a therapeutic compound, an organic compound, an inorganic compound, a nucleic acid, an oligonucleotide (oligo), a protein, a biological molecule, a macromolecule, lipid, oil, fillers, solution, a cell or a tissue. Agents include DHEAS, and pharmaceutically or veterinarily acceptable salt thereof. Agents may be added to prepare a formulation comprising an active compound and used in a formulation or a kit in a pharmaceutical or veterinary use.

The term “airway”, as used herein, means part of or the whole respiratory system of a subject which exposes to air. The airway includes, but not exclusively, throat, windpipes, nasal passages, sinuses, a respiratory tract, lungs, and lung lining, among others. The airway also includes trachea, bronchi, bronchioles, terminal bronchioles, respiratory bronchioles, alveolar ducts, and alveolar sacs.

The term “carrier”, as used herein, means a biologically acceptable carrier in the form of a gaseous, liquid, solid carriers, and mixtures thereof, which are suitable for the different routes of administration intended. Preferably, the carrier is pharmaceutically or veterinarily acceptable.

The composition may optionally comprise other agents such as other therapeutic compounds known in the art for the treatment of the condition or disease, antioxidants, flavoring agents, coloring agents, fillers, volatile oils, buffering agents, dispersants, surfactants, RNA inactivating agents, propellants and preservatives, as well as other agents known to be utilized in therapeutic compositions.

“An effective amount” as used herein, means an amount which provides a therapeutic or prophylactic benefit.

A “composition for inhalation” as used herein is a mixture of chemical compounds that can be introduced into the animal or human patient through the respiratory system, including nasally or orally.

### Compositions

One aspect of the invention is a composition for inhalation comprising a divalent cation and an aqueous suspension of DHEAS. The composition for inhalation can be used for administration to patients for the treatment of a respiratory disease or condition.

Dehydroepiandrosterone sulfate, 5-Androsten-3β-ol-17-one sulfate, (DHEAS) is the sulfate form of DHEA. Dehydroepiandrosterones are non-glucocorticoid steroids. Both DHEA, also known as prasterone or 5 androsten-3 beta-ol-17-one, and DHEAS, are endogenous hormones secreted by the adrenal cortex in primates and a few non-primate species in response to the release of adrenocorticotropic hormone (ACTH). DHEA is a precursor of both androgen and estrogen steroid hormones important in several endocrine processes. DHEA is thought to have a role in levels of DHEA in the central nerve system (CNS), and in psychiatric, endocrine, gynecologic, obstetric, immune, and cardiovascular functions. DHEAS or its pharmaceutically acceptable salts are believed to improve uterine cervix maturation and uterine musculature sensitivity to oxytocin in late phase pregnancy. DHEAS and its pharmaceutically acceptable salts are thought to be effective in the therapy for dementia, for the therapy of hyperlipemia, osteoporosis, ulcers, and for disorders associated with high levels of, or high sensitivity to adenosine, such as steroid-dependent asthma, and other respiratory and lung diseases. Dehydroepiandrosterone itself was administered intravenously previously, subcutaneously, percutaneously, vaginally, topically and orally in clinical trials. DHEAS is a sulfate, which can exist as a protonated form or as a salt, associated with a cation. DHEAS sodium salt can exist in powder form as the anhydrous form, and as a crystalline dihydrate form. The anhydrous form was found to absorb water and convert to a hydrated form under conditions of normal humidity. It is generally desired that the cation be veterinarily or pharmaceutically acceptable.

The compositions of the present invention may have more than one cation present in the aqueous suspension of DHEAS. For instance, the composition may be prepared by combining a solution of DHEAS sodium salt with a solution containing the divalent cation. Under these conditions, both sodium and the divalent cation would be present in composition. Combinations of divalent cations may also be used.

The ions of the compositions of the invention, including the divalent cations and DHEAS in solution can be completely solvated and unassociated, or can exist as ion pairs. DHEAS, when dissociated, will generally exist in aqueous solution as an anion. DHEAS as used in the invention in aqueous solution or suspension can either be protonated, or can be associated with a cation. An ion pair is a pair of oppositely charged ions held together by Coulomb attraction without formation of a covalent bond. Experimentally, an ion pair behaves as one unit in determining conductivity, kinetic behavior, osmotic properties, etc. An ion pair, the constituent ions of which are in direct contact (and not separated by an intervening solvent or other neutral molecule) is designated as a ‘tight ion pair’ (or ‘intimate’ or ‘contact ion pair’). By contrast, an ion pair whose constituent ions are separated by one or several solvent or other neutral molecules is described as a ‘loose ion pair’. The members of a loose ion pair can readily interchange with other free or loosely paired ions in the solution.

The pH of the compositions of the invention are generally near neutral pH (pH 7). It would be understood in the art that where the pH is either too acidic or too basic, there would be irritation to the respiratory system on contact with the compositions. In some embodiments the pH is about 7. In some embodiments, the pH is between about 6.5 and 7.5; in some embodiments the pH is between about 6 and 7.5; in some embodiments; the pH is between about 6 and 8, in some embodiments; the pH is between about 5 and 8; in some embodiments, the pH is between about 5 and 9; in some embodiments, the pH is between about 4 and 10. To ensure that pH can be maintained in a distinct range, a suitable pharmaceutically acceptable buffer system can be used. To adjust the pH, also acids or bases can be used

In some cases, the DHEAS will associate with or form a complex with the divalent cation which is less soluble than the DHEAS sodium salt. In aqueous solution, the solubility of DHEAS-Na is about 17 mg/ml and the solubility of DHEAS-Na+Mg2+ is about 0.7 mg/ml.

The molar amount of the divalent cation in the compounds of the present invention are usually on the same order as the molar amount of the DHEAS. The divalent cations are not, for instance, present only in trace amounts. In some embodiments, the molar ratio of divalent cation to DHEAS is about 0.5, 0.75, 0.9, 1, 1.1, 1.25, 1.5, 2, 4, and 5. In some embodiments the range is between about 0.1 and 5, in some embodiments the range is between about 0.2 and 5, in some embodiments the range is between about 0.25 and 4, in some embodiments the range is between about 0.5 and 2, in some embodiments the range is between about 0.75 and 1.25, in some embodiments the range is between about 0.9 and 1.1.

The amount of DHEAS in the aqueous suspension must be enough to be therapeutically effective when administered to a patient as an aerosol. The amount should not be so high that the viscosity, flow properties, and stability of the suspension are compromised. It can be convenient to express the amount of DHEAS in the aqueous suspension as a weight percent based on the weight of DHEAS sodium salt. In some embodiments, the amount of DHEAS is about 0.1, 0.25, 0.5, 0.75, 1, 1.5, 2, 2.5, 3, 3.5, 4, 4.5, 5, 5.5, 6, 7, 8, 9, 10, 12, 15 weight percent of the weight of the aqueous suspension based on the weight of DHEAS sodium salt. In some embodiments the amount of DHEAS is about 2 weight percent of the weight of the aqueous suspension based on the weight of DHEAS sodium salt, in some embodiments the amount of DHEAS is about 2.5 weight percent of the weight of the aqueous suspension based on the weight of DHEAS sodium salt, in some embodiments the amount of DHEAS is about 3 weight percent of the weight of the aqueous suspension based on the weight of DHEAS sodium salt, in some embodiments the amount of DHEAS is about 3.5 weight percent of the weight of the aqueous suspension based on the weight of DHEAS sodium salt, in some embodiments the amount of DHEAS is about 4 weight percent of the weight of the aqueous suspension based on the weight of DHEAS sodium salt. In some embodiments, the range of DHEAS amounts is from 0.25 to 5, 0.5 to 5, 0.75 to 4, or 2 to 4 weight percent of the weight of the aqueous suspension based on the weight of DHEAS sodium salt.

A suspension as used herein refers to a two-phase system consisting of a finely divided separate phase dispersed in a liquid, or gas. The separate phase is generally a solid, but could also be a liquid. The size of the particles in the suspension can vary over a wide range from colloidal particles to macroscopic particles. For inhalation applications, it is generally preferred that the particles be small enough to be effectively carried into the respiratory system. It is also generally preferred that the particles do not rapidly settle and can be easily redispersed. The suspension of DHEAS of the present invention generally has DHEAS that is in a finely dispersed phase consisting of respirable particles. In some embodiments, 90 volume % have a diameter of less than 5 μm, more preferably less than 3 μm. In some embodiments, 50 volume % have a diameter of less than 2.5 μm, more preferably less than 1.5 μm. The finely dispersed DHEAS may be associated with a cation, or may be protonated. In general, some of the finely dispersed DHEAS will be associated with a divalent cation. In the composition for inhalation, some DHEAS may remain dissolved in the aqueous solution.

A key ingredient in the inhalation compositions is water. It is used both as a vehicle and as a solvent for the other agents and ingredients. Water is desirable as part of the composition for inhalation due in part to its inertness, liquidity, low viscosity, tastelessness, freedom from irritating qualities, and lack of pharmacological activity. The water used in the inhalation formulations of the invention must be in a purified form. Such water may be prepared by distillation, by use of ion-exchange resins, or by reverse osmosis. A wide variety of commercially available stills can be used to produce distilled water. Such water may be sterile. Quality-control procedures for monitoring the microbiological quality of water should be performed in the pharmaceutical manufacturer's production facilities. Ion-exchange (deionization, demineralization) processes can be used to remove most of the major impurities in water efficiently and economically. The major impurities in water are often calcium, iron, magnesium, manganese, silica, and sodium. The cations usually are combined with the bicarbonate, sulfate, or chloride anions. Hard waters are those that contain calcium and magnesium cations. Bicarbonates are the major impurity in alkaline waters. Ultraviolet radiant energy (240 to 280 nm), heat, or filtration can be used to limit the growth of, kill, or remove microorganisms in water. Reverse osmosis can also be used to purify water using semipermeable membranes, for example, to remove organic molecules. Viruses and bacteria can generally be removed by filtration. Frequently, two or more methods are used to produce the water desired, for example, filtration and distillation, or filtration, reverse osmosis, and ion exchange.

The compounds of the invention may also contain one or more excipients. Excipients are generally inert substances that act as a vehicle, a diluent, or assist in the delivery of a drug. In some cases, the excipients can provide a taste masking or sweetening function. A suitable excipient is one selected from lactose, dextran, galactose, D-mannose, sorbose, trehalose, sucrose, raffinose, xylitol, sorbitol, mannitol, magnesium sulfate, magnesium aspartate, magnesium-gluconate, L-lysine, L-arginine, glycerin, glycerol, xylitol, sorbitol, mannitol, and a mixture thereof. In some embodiments, xylitol is used as the excipient. The amount of excipient added on a weight basis is on the same order as the weight of DHEAS based on the weight of the DHEAS sodium salt. In some embodiments, the weight ratio of excipient to DHEAS is about 0.1, 0.2, 0.25. 0.5, 0.75, 0.9, 1, 1.1, 1.25, 1.5, 2, 4, 5, and 10. In some embodiments the range is between about 0.1 and 10, in some embodiments the range is between about 0.2 and 5, in some embodiments the range is between about 0.25 and 4, in some embodiments the range is between about 0.5 and 2, in some embodiments the range is between about 0.75 and 1.25.

In some embodiments sweetening agents are added to improve the properties of the suspension as an aerosol. In some cases, the excipients described above are sugars or sugar alcohols that provide sweetening. In some cases, the addition of additional sweetening agents make the formulation more palatable to the patient. It can be useful to employ a high intensity sweetener that provides a high amount of sweetness per weight. High intensity sweetener generally means a sweetener that provides at least about 2 g of sucrose equivalent sweetness per gram sweetener. In some cases, a high intensity sweetener can provide about 40 g of sucrose equivalent sweetness per gram and in some cases about 200 g of sucrose equivalent sweetness per gram. One gram of certain high intensity sweeteners, e.g., neotame, can provide the sweetness of about 8,000 g sucrose. Many high intensity sweeteners are known to those skilled in the art. Those that can be used in the present invention include aspartame, acesulfame, saccharine, cyclamate, neotame, sucralose, brazien and other protein based sweeteners, plant extracts, such as, stevia and luo hon guo, and the various salts, derivatives, and combinations or mixtures thereof. In some embodiments, sodium saccharine is used as the sweetening agent.

In some embodiments, mint flavoring agents, such as menthol (also referred to as levomenthol), are used

In some embodiments, the compositions further comprise an emulsifier or surfactant. The emulsifier or surfactant can act to stabilize the aqueous suspension of active ingredient. In some embodiments, the emulsifier or stabilizer comprises Polysorbate 80/Tween® 80 (PS80), Poloxamer 188/Lutrol® F68 (P188), Poloxamer 407/Lutrol® F127 (P407), Vitamin E-TPGS (TPGS), or hydroxypropylmethylcellulose (HPMC). In some embodiments, the emulsifier is Vitamin E-TPGS.

Viscosity agents such as natural gums (eg, acacia, xanthan and cellulose derivatives, such as sodium carboxymethylcellulose and hydroxypropylmethylcellulose, may be used at low concentrations (<0.1%) to function as protective colloids, but at higher concentrations they can then function as viscosity-increasing agents and decrease the rate of settling of deflocculated particles or provide stability in a flocculated suspension. Those with skill in the art will understand that in some cases, it can be undesirable to add agents which increase the viscosity of the formulation because nebulization may be negatively affected, for example the inhalation time may be prolonged.

Buffers may be included in the formulation, for instance, if the drug has ionizable groups in order to maintain a low solubility of the drug. Buffers also may be included to control the ionization of preservatives, ionic viscosity agents, or to maintain the pH of the suspensions within a suitable range.

The formulations of the invention may contain other drugs, e.g., combinations of therapeutic agents may be processed together. The combination of drugs will depend on the disorder for which the drugs are given, as will be appreciated by those in the art.

### Methods

One aspect of the invention is a method of making a composition for inhalation comprising the steps of: dispersing DHEAS in a first aqueous volume, mixing a compound comprising a divalent cation in a second aqueous volume: and combining the aqueous volumes to form a suspension of DHEAS. This method allows for the formation of a fine suspension of DHEAS in aqueous solution.

In some embodiments of the present invention the DHEAS sodium salt is the form used to introduce DHEAS into the first aqueous volume. The sodium salt is desirable, in that the salt is generally pharmaceutically acceptable. Other salts of DHEAS such as the lithium, potassium, or ammonium salts may also be used. DHEAS could also be dissolved in its protonated form in acidic solution.

The mixing of the DHEAS and other solutes described herein can generally be accomplished by adding the solute to water or an aqueous mixture and stirring with or without the addition of heat. In some cases, raising the temperature of the water or aqueous solution can increase the rate of mixing or dissolution. The mixing or dissolution can be facilitated by raising the temperature 5° C., 10° C., 15° C., 20° C., or 30° C. over room temperature. It will be understood by those skilled in the art that if the temperature is raised too high for to long a time, there is a risk of degradation to the compounds in the formulation.

In some embodiments, the compounds that are mixed into the composition are dissolved so as to form a solution. A solution is a mixture that can be prepared by mixing a solid, liquid, or gas in another liquid and represents a group of preparations in which the molecules of the solute or dissolved substance are dispersed among those of the solvent. In some cases the solutions will be homogeneous solution. A homogeneous aqueous solution will generally be clear, indicating that there are few or no aggregates that are large enough to scatter light. A homogeneous solution, in some cases, need not be completely molecularly dissolved, and for instance there may be some aggregation of the solutes in the solution. Some of the compounds in the composition may not be completely dissolved in solution, and may be partly or completely in a solid, semi-solid, or liquid form in suspension. In a suspension, some components may be completely dissolved, while other components are partly or completely undissolved.

An aqueous suspension is a suspension wherein the solution, or liquid continuous phase, contains water. In most aqueous solutions or suspensions, the solvent is mainly water. The aqueous solutions or suspensions may also contain other co-solvents that are soluble in water. The co-solvents are generally solvents that are at least partly soluble in water including alcohols such as, ethanol. In some cases, the co-solvent can be removed before the composition is provided to patients, and in other cases, the co-solvent will remain in the aqueous solution. Where the aqueous solvent remains in the composition inhaled by patients, it would be understood in the art that the solvent must be veterinarily or pharmaceutically acceptable.

While, as described above, the aqueous suspension of DHEAS generally has a pH near neutral pH, the pH of the first and second aqueous volumes need not be near neutral. In some cases the pH of the first and second volumes can be adjusted, for instance to improve solubility of one or more ingredients. If the mixing of the first and second aqueous phases results in a pH that is different from the desired pH, e.g. away from neutral pH, the pH of the resulting composition can be adjusted by the addition of acid or base, and/or by using a buffer.

One aspect of the present invention is the mixing of the first and second aqueous volumes to form a suspension of DHEAS. In some embodiments it is desirable to mix the solutions in a controllable manner. Mixing in a controllable manner can affect the particle size of the suspension that is formed. In some embodiments, it is desirable to add the second aqueous solution to the first aqueous solution in a controllable manner. One aspect of adding in a controllable manner is controlling the rate at which the volumes are combined. Adding in a controllable manner can involve slowly adding one solution to the other solution with agitation. The addition of one solution slowly to the other solution with agitation can result in a small and consistent particle size for the suspension. The addition can take place over a period of minutes or hours. In some embodiments, the addition takes place over 10 min, 20 min, 30 minutes, 40 minutes, 60 minutes, 90 minutes, 2 hours, 3 hours, 4 hours, 6 hours, or 8 hours. The agitation can be accomplished, for example, by stirring using magnetic stirring or stirring with a paddle type stirring apparatus.

While the control of the mixing of the first and second volumes can produce a fine suspension, in some cases it is desirable to process further in order to refine the suspension by appropriate technologies. In some cases, impeller types of equipment can be used, but in some cases, further reduction in particle size can be accomplished with an ultraturax, or high pressure homogenizer. The initial suspension created on mixing of the fluid volumes may be subjected to high pressure homogenization by passage of the suspension between a finely ground valve and seat high pressure. This, in effect, produces an atomization that is enhanced by the impact received by the atomized mixture as it strikes the surrounding surfaces. The homogenizer can operate at pressures of, for example, 1,000 to 30,000 psi and can produce fine dispersions. Different valve assemblies, two-stage valve assemblies, and equipment with a wide range of capacities may be used. A two-stage homogenizer is typically constructed so that the liquid aqueous formulation after treatment in the first valve system, is conducted directly to another where it receives a second treatment. The machine may be equipped with a pump that carries the liquid through the various stages of the process. For small-scale preparations a hand-operated homogenizer may be used. A homogenizer generally does not incorporate air into the final product. The suspensions may be homogenized using ultrasonic devices. For example, an oscillator of high frequency (100 to 500 kHz) is connected to two electrodes between which is placed a piezoelectric quartz plate. While the oscillator is operating, high-frequency waves flow through the fluid. The suspensions may be homogenized using a microfluidizer, which subjects the suspension to an extremely high velocity in an interaction chamber; as a result water insoluble particles are subjected to shear, impact, and cavitation.

One aspect of the invention is a method of making a composition for inhalation comprising the steps of; mixing DHEAS sodium salt, an excipient, a stabilizing agent, and a sweetening agent in an first aqueous volume; mixing a compound comprising magnesium chloride in a second aqueous volume: mixing the first and second aqueous volumes to form a suspension of DHEAS; and homogenizing the suspension. In this aspect, the DHEAS sodium salt, the excipient, the preservative, and the sweetening agent are all mixed in a first aqueous volume. In some embodiments a buffer is included in the first and/or in the second aqueous volume.

In some embodiments, each of the ingredients is added and mixed separately. In some cases, two or more ingredients may be mixed together. The temperature may be raised or lowered during mixing, for example, to aid in dissolution or mixing of the ingredients. In this aspect, the compound comprising the divalent cation, e.g. magnesium chloride, is mixed into the second aqueous volume. In some embodiments, after mixing, the magnesium chloride dissolves to form a homogeneous solution, which may, for example, on visual inspection, have a clear appearance. The first and second aqueous volumes are mixed, usually in a controlled manner. In one embodiment, the second aqueous volume is added in a controlled manner to the agitated first aqueous volume. The addition can take place over a period of minutes or hours. In some embodiments, the addition takes place over 10 min, 20 min, 30 minutes, 40 minutes, 60 minutes, 90 minutes, 2 hours, 3 hours, or 4 hours. The agitation can be accomplished, for example, by stirring using magnetic stirring or stirring with a paddle type stirring apparatus. The suspension may be homogenized as previously described.

### Uses

The compositions of the present invention is designed to be aerosolized by nebulizers for administration via the nose or mouth into the respiratory tract of humans or animals. Administration by inhalation can allow high concentrations of drug to be delivered effectively into the upper and lower respiratory tract resulting in rapid deposition of a therapeutically effective dose into the upper or lower respiratory tract. This mode of administration allows a drug targeting bringing the drug to the site where needed in the body to treat a disease and avoiding by this firm of administration high drug absorption and systemic drug levels which may cause undesired side effects. Hence, systemic side effects can be significantly reduced or completely avoided. Drugs can be inhaled as aerosols, which are airborne suspensions of fine particles. The particles can be comprised of either liquid droplets, or solids that remain suspended long enough to permit deposition deep into the lungs. The effects produced by the inhaled particles depend on their solubility and particle size. The size of the aerosol droplets or particle-containing solution can be between 1 and 5 μm in diameter to permit the medication to reach both, the central and peripheral lungs including the bronchopulmonary mucosal surface. Particles larger than 3 μm rarely reach the alveoli, where the conditions for absorption are greatest; particles below about 1 μm are generally exhaled without deposition in the lungs. Lung deposition is primarily triggered by the particle size and the inhalation patterns. Inhalation devices that emit aerosolized particles at a high velocity (e.g., pressurized MDIs, or pMDIs) may lead to a high degree of drug deposition in the oropharynx. The high velocity of aerosols can make it difficult to coordinate inhalation with device actuation, and the inability to coordinate inhalation with actuation can result in the deposition of drug in the oropharynx. Reducing the speed of the aerosol particles can improve delivery of the drug into the airways. In addition, decreasing size of the aerosol particles can improve drug delivery. Administration of the inventive aqueous formulation can be best achieved by nebulization via for instance a jet- or vibrating membrane nebulizers. For lung deposition via oral inhalation or a face mask an electronic nebulizer generating the aerosol via a perforated vibrating membrane (eFlow®, PARI Pharma GmbH) is preferred and characterized by a respirable fraction (drug in droplets <5 μm) of >50%, a mass median aerodynamic diameter (MMAD) between 2 and 5 μm and more preferably 3-4 μm and a geometric standard deviation <2. Furthermore, the nebulizer is characterized that the delivered dose (DD) exiting the mouthpiece or a face mask under simulated breathing conditions according example 3 is >50% of the nominal dose. For administration of the inventive DHEAS formulation into the upper respiratory tract, such as the nose or paranasal cavities either a jet or vibrating membrane nebulizer can be used. Alternatively, an atomizer in form of a nasal pump spray may be applicable if drug deposition into the nasal cavity is the primary target to treat for instance allergic or non allergic rhinologic diseases, such as hayfever, rhinitis or sinusifis.

The use of the inhalation route allows easy accessibility to the respiratory tract because the DHEAS and other co-therapeutic agents can be directly administered to sites of action in the lungs or upper respiratory tract such as the nose or paranasal cavities. Advantages of inhalation include: (i) medication is delivered directly to the target site; (ii) small amounts of drug suffice to prevent or treat symptoms; (iii) adverse reactions can be much less than those produced by systemic administration; and (iv) there is a rapid and predictable onset of action.

The compositions of the present invention can be administered using nebulizers. Inhalation nebulizers deliver therapeutically effective amounts of pharmaceuticals by forming an aerosol consisting of droplets in a selected size range which carry the particles of a distinct size either to the upper and/or lower respiratory tract. It is apparent, that the size of the particles must be smaller than the size of the droplets to secure that all drug particles can be carried facilitating deposition to the designated target site. Furthermore, when using a perforated vibrating membrane nebulizer it is desired that the majority of particles is smaller than 3 μm to avoid that particles may be sieved out. Nebulizer systems offer the advantage over metered dose inhalers (MDIs) and dry powder inhalers (DPIs) that the drug can be administered via spontaneous tidal breathing, and no complex co-ordination by the patient is needed. This feature facilitates drug deposition to the target site in a more reliable way than for MDIs and DPIs and reducing the failure rate compared to these inhalation delivery systems requiring complex inhalation patterns. Since the drug is delivered in many consecutive breathing cycles and not as a single or dual shot bolus as characteristic for MDIs and DPIs, a more reliable drug deposition to the target site in the lungs can be achieved. With nebulizers, drugs can be mixed and administered at the same time if the chemical and physical compatibility of drugs and formulations have been verified beforehand. A variety of inhalation nebulizers are known. In jet nebulizers, the aerosol is formed by a high-velocity airstream from a pressurized source directed against a thin layer of liquid solution. Also, for example, EP 0 170 715 A1 uses a compressed gas flow to form an aerosol. A nozzle is arranged as an aerosol generator in an atomizer chamber of the inhalation nebulizer and has two suction ducts arranged adjacent a compressed-gas channel. When compressed air flows through the compressed-gas channel, the liquid to be nebulized is drawn in through the suction ducts from a liquid storage container. This nebulizer is representative of continuously operating inhalation nebulizers, in which the aerosol generator produces an aerosol not only during inhalation but also while the patient exhales. The compositions of the present invention can be administered using nebulizers that utilize other means of aerosol generation such as an oscillating aerosol generator including a vibrating diaphragm. (see Knoch M. & Keller M.: The customized electronic nebuliser: a new category of liquid aerosol drug delivery systems. Expert Opinion Drug Deliv. 2005, 2(2), 377-390). The inventive DHEAS formulation and potential compositions with other drugs are suitable for administration with nebulizers, aerosol generators, or fluid droplet production apparatus such as, for example those described in U.S. Pat. No. 6,962,151, U.S. Pat. No. 6,938,747, U.S. Pat. No. 7,059,320, U.S. patent application Ser. No. 10/810,098, U.S. patent application Ser. No. 10/522,344, U.S. patent application Ser. No. 10/533,430.

One aspect of the invention is the administration of the compositions of the present invention with portable, battery-powered nebulizers, such as the eFlow® (PARI Pharma GmbH) electronic nebulizer (Keller M. et al.: Nebulizer Nanosuspensios: Important Device and Formulation Interactions Proceedings Respiratory Delivery VIII, 2002, 197-205). Portable nebulizers make it easier for actively mobile patients to use the inhalation compositions.

The compositions and methods of the present invention can be used to treat respiratory diseases such as those diseases or conditions related to the respiratory system. Examples include, but not limited to, airway inflammation, allergy(ies), asthma, impeded respiration, cystic fibrosis (CF), Chronic Obstructive Pulmonary Diseases (COPD), allergic rhinitis (AR), Acute Respiratory Distress Syndrome (ARDS), pulmonary hypertension, airway inflammation, bronchitis, airway obstruction, bronchoconstriction, microbial infection, lung cancer, and viral infection, such as SARS.

### Combination Therapy

One aspect of the invention is the co-administration of DHEAS as a composition for inhalation as described herein in combination with another respiratory therapeutic agent in order provide an overall benefit the patient. One advantage of using the compositions is the compliance by the patients in need of such prophylaxis or treatment. Respiratory diseases such as asthma or COPD are multifactoral with different manifestations of signs and symptoms for individual patients. As such, most patients are treated with multiple medications to alleviate different aspects of the disease. A fixed combination of the first active agent, such as DHEA-S, and the second active agent, such as described below, permits more convenient yet targeted therapy for a defined patient subpopulation. Patient compliances can, for example, be improved by simplifying therapy and by focusing on each patient's unique disease attributes so that their specific symptoms are addressed in the most expeditious fashion. Further, there can be the added advantage of convenience or savings in time in the administering of both the first and second active agents in one administration.

In some cases, the DHEAS and the other therapeutic agent are both administered by inhalation. In other cases, the DHEAS is administered by inhalation as described herein, and the other therapeutic agent is administered by other means such as buccal, oral, rectal, vaginal, nasal, intrapulmonary, ophthalmic, optical, intracavitary, intratraccheal, intraorgan, topical (including buccal, sublingual, dermal and intraocular), parenteral (including subcutaneous, intradermal, intramuscular, intravenous and intraarticular) and transdermal administration. Co-administration may include administering the DHEAS and the other agent at the same time, and may involve administering the DHEAS and the other agent at different times.

In some embodiments the compositions of the invention provide aerosol formulations comprising a combination of DHEAS and an anti-muscarinic agent. Treatment of respiratory conditions and diseases with a combination of DHEA derivatives and an antimuscarinic agent is described in WO 04/014293 incorporated herein by reference. Examples of suitable anti-muscarinic agents include ipratropium and oxitropium bromide, tiotropium bromide, and troventol.

In some embodiments the invention provides methods for treating a human or animal comprising administering a DHEAS composition for inhalation as described herein and a beta-2 agonist bronchodilator. Suitable beta-2-agonist bronchodilators include albuterol (synonym salbutamol), terbutalin, levalbuterol, formoterol, and salmeterol either as a free bases or pharmaceutically acceptable salt. The treatment of respiratory conditions and diseases with a combination of DHEA derivatives and beta-agonist bronchodilators is described in WO 05/011603 incorporated herein by reference. Other examples of long and short lasting beta.2 agonists are ephedrine, isoproterenol, isoetharine, epinephrine, metaproterenol terbutaline fenoterol, procaterol, albuterol, levalbuterol, formoterol bitolterol and bambuterol, in any acceptable pharmaceutical salt form or as an isomer or entaniomer. Water stable salts and/or aqueous formulations of the long-acting beta.2-agonist such as carbuterol, indacaterol, salmeterol formoterol and compatible with the inventive DHEAS formulation are preferred.

In some embodiments the invention provides methods for treating a human or animal comprising administering a DHEAS composition for inhalation as described herein and a leukotriene receptor antagonist. Treatment of respiratory conditions and diseases with a combination of DHEA derivatives and a leukotriene receptor antagonist is described in WO 05/011595 incorporated herein by reference. Examples of leukotriene receptor agonists include montelukast, zafirlukast and pranlukast.

In some embodiments the invention provides methods for treating a human or animal comprising administering a DHEAS composition for inhalation as described herein and a PDE-4 inhibitor. Treatment of respiratory conditions and diseases with a combination of DHEA derivatives and a PDE-4 inhibitor is described in WO 05/011602 incorporated herein by reference. Examples of PDE-4 inhibitors include roflumilast (Altana Pharma, Germany), and cilomilast (Ariflo™, SB 207499, SmithKline Beecham).

In some embodiments the invention provides methods for treating a human or animal comprising administering a DHEAS composition for inhalation as described herein and an antihistamine. Treatment of respiratory conditions and diseases with a combination of DHEA derivatives and an antihistamine is described in WO 05/011604 incorporated herein by reference. Examples of suitable antihistamines include cetirizine hydrochloride, which is commercially available as orally administered Zyrtec® tablets and syrup (Pfizer Inc., New York, N.Y.), loratadine, which is commercially available as orally administered Claritin-D 12 Hour Extended Release Tablets (Schering Corporation, Kenilworth, N.J.), desloratadine, which is commercially available as orally administered Clarinex®, and fexofenadine hydrochloride, which is commercially available as orally administered Allegra® capsules and tablets (Aventis Pharmaceuticals Inc., Kansas City, Kans.).

In some embodiments the invention provides methods for treating a human or animal comprising administering a DHEAS composition for inhalation as described herein and a lipoxygenase inhibitor. Treatment of respiratory conditions and diseases with a combination of DHEA derivatives and a lipoxygenase inhibitor is described in WO 05/011613 incorporated herein by reference. Examples of lipoxygenase inhibitors include zileuton, which is currently commercially available as Zyflo™, Tablets (Abbott Laboratories, North Chicago, Ill.) These are oral drugs only and may require complex formulation technologies, there is not hint these drugs will be feasible with the inventive DHEAS formulation.

In some embodiments the invention provides methods for treating a human or animal comprising administering a DHEAS composition for inhalation as described herein and a tyrosine kinase inhibitor such as described in U.S. Pat. No. 6,169,091, a delta opioid receptor antagonist as described in U.S. Pat. No. 6,514,975, a neurokinin receptor antagonist as described in U.S. Pat. Nos. 6,103,735; 6,221,880; and, 6,262,077, or a VCAM inhibitor as described in U.S. Pat. Nos. 6,288,267; 6,423,728; 6,426,348; 6,458,844; and, 6,479,666. Treatment of respiratory conditions and diseases with a combination of DHEA derivatives and a tyrosine kinase inhibitor, delta opioid receptor antagonist, neurokinin receptor antagonist, or VCAM inhibitor is described in WO 05/011594 incorporated herein by reference.

In some embodiments the invention provides methods for treating a human or animal comprising administering a DHEAS composition for inhalation as described herein and a methylxanthine derivative. Treatment of respiratory conditions and diseases with a combination of DHEA derivatives and a methylxanthine derivative is described in WO 05/011608 incorporated herein by reference. An example of methylxanthine derivatives is theophyllin, which is commercially available as Theo-Dur (Schering Corp., Kenilworth, N.J.), Respbid, Slo-Bid (Rhone-Poulenc Rorer Pharmaceuticals Inc., Collegevilla, Pa.), Theo-24, Theolair, Uniphyl, Slo-Phyllin, Quibron-T/SR, T-Phyl, Theochron, and Uni-Dur.

In some embodiments the invention provides methods for treating a human or animal comprising administering a DHEAS composition for inhalation as described herein and a cromone. Treatment of respiratory conditions and diseases with a combination of DHEA derivatives and a cromone is described in WO 05/011616 incorporated herein by reference. Examples of a cromone include cromolyn sodium or nedocromil sodium. Nedocromil sodium is commercially available in Australia as Tilade® CFC-Free (Aventis Pharma Pty. Ltd., Australia). Cromolyn sodium is commercially available as Intal® (Rhone-Poulenc Rorer Pharmaceuticals Inc., Collegevilla, Pa.).

In some embodiments the invention provides methods for treating a human or animal comprising administering a DHEAS composition for inhalation as described herein and an anti-Ig-E antibody. Treatment of respiratory conditions and diseases with a combination of DHEA derivatives and an anti-Ig-E antibody is described in WO 15/11614 incorporated herein by reference. An exemplary anti-IgE antibody is E-25, omalizumab, which is available as Xolair® (Genentech, Novartis).

In some embodiments the invention provides methods for treating a human or animal comprising administering a DHEAS composition for inhalation as described herein and a glucocorticosteroid. Treatment of respiratory conditions and diseases with a combination of DHEA derivatives and a glucocorticosteroid is described in WO 05/099720 incorporated herein by reference. Examples of suitable glucocorticosteroids include beclomethasone propionate, budesonide, flunisolide, fluticasone propionate, triamcinolone acetonide, and ciclesonide. These compounds were not tested and may interfere with DHEAS formulation.

### EXAMPLES

### Example 1

### Preparation of DHEAS Suspension for Inhalation

First, a cleaned 20 L Duran flask is sterilized with dry heat (180° C./30 min.). To the other is added about 1,7644 g of purified water. Next, about 120 g 1M hydrochloric acid (HCL) are added to the DURAN flask, followed by the addition of about 600 g xylitol. Magnetic stirring is carried out until the xylitol is visibly dissolved after which the following materials are added one after the other to the DURAN flask: about 6 g of propyl-4-hydroxybenzoate sodium, about 14 g of methyl-4-hydroxybenzoate sodium, and about 10 g of saccharin sodium hydrate. Magnetic stirring is continued until all compounds are visibly dissolved after which the solution is heated to a temperature of 35-40° C. while magnetically stirred. When the solution reaches a temperature of 30° C., and about 60 g of Vitamin E TPGS are added to the DURAN flask. When the temperature of the solution reaches 35° C., about 6 g of levomenthol are added to the DURAN flask. The flask is magnetically stirred at 35 40° C. until the Vitamin E TPGS and the levomenthol are visibly dissolved. After dissolution, the solution is cooled to a temperature below 30° C., after which about 700 g of DHEAS sodium H₂O (DHEAS) are added to the DURAN flask and magnetically stirred overnight (if necessary, a paddle stirrer can be used).

In a separate vessel, about 340 g magnesium chloride-H₂O is added to about 500 g of purified water and dissolved by slight agitation until visibly dissolved. The magnesium chloride-H₂O solution is added gradually and slowly to the DURAN flask while stirring with the magnetic stirrer and paddle mixer. After completion of the addition, the suspension is stirred for 30 min at 20-25° C.

A 10 ml sample is taken via syringe. The pH value is determined, and if necessary, the pH is adjusted to pH 7.0±0.3 by use of 1M hydrochloric acid or 1M sodium hydroxide solution, respectively.

The suspension may be transferred to a high pressure homogenizer for homogenization. After cleaning the equipment and autoclaving the tubing, the high pressure homogenization is commenced. The suspension is homogenized discontinuously under cooling at 1500 bar for 5 cycles with a Microfiuidizer M110 EH2 high pressure homogenizer. At the completion of the high pressure homogenization, the suspension may be dispensed, for instance into sterile bottles or tubes or other containers for storage, transport, and/or dispending. The suspension thus prepared may be administered with no further processing, for instance, with an ultrasonic nebulizer.

In some cases, the mixture (prior to the addition of DHEAS) is recirculated through the homogenizer while DHEAS was slowly added to the holding tank. Homogenization is then continued for another 60 minutes with recirculation of the DHEAS-containing mixture prior to the addition of the Magnesium chloride solution. In other cases, DHEAS and Magnesium chloride are added to the holding tank and then the suspension is recirculated through the homogenizer for 60 minutes. Additional modifications to flow rates, pressures and piping were also made.

### Example 2

### Treatment of Asthmatic Patients

Thirty seven patients ages 18 to 33 are diagnosed with chronic asthma. Each of the patients is treated by inhalation of about 35 mg of DHEAS twice daily. The DHEAS is administered using and eFlow® nebulizer (PARI Pharma GmbH) using about 0.5-31 mL of a 3.5% DHEAS suspension as described in Example 1. After 2, 4, 8 and 12 weeks the patients are monitored to determine the efficacy of the treatment. Efficacy is determined by one or all of: (1) mean changes from baseline in daytime and nighttime asthma symptom scores over the 12 week treatment phase where the symptom scores are based on the subjective evaluation by the patients or their parents based on a 0-3 rating system in which 0=no symptoms, 1=mild symptoms, 2=moderate symptoms, and 3=severe symptoms. (2) spirometry test variables, including FEV.sub.1, FEF.sub.25-75 (forced expiratory flow during the middle half of the forced vital capacity in liters per second) and FVC (forced vital capacity in liters), performed at clinic visits in the subset of patients capable of performing spirometry testing; (3) PEF (peak expiratory flow in liters per minute); (4) differences in asthma-related health care utilization and indirect health care costs. Improvements in the efficacy measures indicates the effectiveness of the inhalation treatment with the DHEAS aqueous suspension.

### Example 3

### Aerosol Characterization of a DHEAS Suspension

This example describes the aerosol characteristics such as particle size distribution and the expected lung dose of a DHEA-S dihydrate suspension (70 mg/2 mL) made as described herein. The concentration of the formulation is 35 mg/ml. The developed suspension has to be nebulized sufficiently and in an acceptable time with an eFlow® electronic nebulizer (PARI Pharma GmbH). The aim for the development was to deliver an in-vivo lung dose of about 20 mg DHEA-S in less than five minutes. This study was carried out with three eFlow® nebulizers (PARI Pharma GmbH) from the upper limit, the middle and lower limit of the specification using the same batch of formulation as in the clinical trial. Particle size determination of the aerosol was carried out by cascade impaction and laser diffraction, delivered dose and nebulization time were determined via breath simulation using standard adult breathing pattern. Respirable dose and in vivo lung dose were calculated from the impactor and breath simulation experiments.

In the breath simulation experiments, one ampoule DHEAS suspension (70 mg/2 mL) was aerosolized within 4.1±0.6 minutes. Using a standard adult breathing pattern of 500 mL tidal volume and 15 breaths per minute, a delivered dose of about 40±3 mg DHEAS was found ex-mouthpiece on the inhalation filter. This means that about 57% of the initially charged drug amount is delivered to the mouth whereas 13% of drug remains in the nebulizer and 30% is being exhaled. Breath simulation experiments were also carried out with placebo formulation in order to determine if there are significant differences regarding the nebulization time. The nebulization time of the placebo was 3.6±0.4 minutes. Comparing the mean results of placebo and verum formulation via t-test or one-way ANOVA, there is no significant difference at the 95% confidence level (P=0.096). However, multifactor ANOVA test considering both factors, device and formulation, reveals a significant difference between placebo and verum (P=0.011). Consequently, formulation type has a significant influence on administration time, but the effect of the device is greater (P=0.004) and covering the formulation's effect. The most important goal of the study was to estimate the lung dose the patients will receive. Generally, particle sizes below 5 μm diameter are regarded respirable. The aerosol produced by the eFlow® has, upon impactor experiments, a mass median diameter of 4.0±0.1 μm and the percentage of particles below 5.0 μm, i.e. the respirable fraction, is 74±3%. The respirable dose, calculated by multiplying the delivered dose with the respirable fraction, is 29 mg of DHEAS. However, it is known from other deposition studies using the eFlow® with radiolabeled formulations that the in-vivo lung dose is only about 60-70% of the in-vitro respirable dose. The main reason for this deviation is probably the dead space of the respiratory tract of typically 150 mL which leads to an increased exhalation of aerosol. The dead volume of the experimental setting is only a few mL and very small compared to the tidal volume of the breathing process. Therefore, the emitted aerosol is collected more effectively on the inspiratory filter than in vivo. Assuming that only 60% to 70% respirable dose will deposit in the lungs, the estimated in vivo lung dose is 17-20 mg of DHEAS.

### Example 4

### Simulated User Tests with eFlow® 30 L (PARI Pharma GmbH) and DHEAS Suspension

The aim of this simulated user tests (SUT) was to study 42 nebulization cycles with eFlow® 30 L and a DHEAS suspension prepared as described herein utilizing the PARI COMPAS breath simulator at standard settings mimicking an adult breathing pattern (15 breaths a 500 ml per min; inhalation:exhalation=1:1). This represents a 6 week therapy with inhalation once daily.

The nebulizer was connected to a sinus pump (PARI breath simulator) mimicking a standard breathing pattern. Inspiratory and expiratory filters are installed between the nebulizer and the pump via a Y-piece. The nebulizer was filled with DHEAS suspension for inhalation comprising 70 mg DHEAS in 2 ml and driven until the end of nebulization. Nebulization can also be interrupted to change saturated filters after suitable time intervals.

Here, 42 cycles of nebulization, cleaning and disinfection with a DHEAS suspension with three different Heads are simulated. Over the 42 cycles an increase of nebulization time up to 10% could be observed. Therefore special cleaning procedures have to be applied. However, the delivered dose was not affected and remained constant.

### Example 5

### Stability of the Suspension Formulation

Samples of the DHEAS suspension prepared as described above are placed for characterization of stability for up to 2 years at three conditions: (1) refrigerated (5° C.), (2) room temperature (25° C.), and (3) accelerated conditions (40° C.). The preliminary data show excellent stability in all parameters of the clinical batch for at least one year under refrigerated conditions. In addition the DHEAS suspension formulation of the invention was found to be stable after 4 weeks room temperature.

The stability of the suspension formulation of the invention is markedly superior to the stability of other DHEAS suspension formulations. For example a saline nebulizer formulation was prepared by adding 0.12% saline (hypotonic saline) to a sterile unit dose glass vial containing 25 mg of powdered DHEA-S. Preliminary stability testing of the saline nebulizer formulation showed that after 24 hours at accelerated temperature or 72 hours at room temperature, the solution deteriorated, became cloudy with precipitate and the concentration of (a degradant) went up.

### Example 6

### Characterization of the DHEAS Suspension

The clinical batch material underwent aerosol characterization with breath simulation and stability testing. Andersen cascade impaction is performed at standard flow rates to quantify the mass of particles at any given size. Andersen cascade impaction is a method used to describe the amount of an aerosol that is potentially available for lung deposition. The results of the Andersen Cascade Impaction at a starting concentration of DHEAS suspension of 70 mg/2 mL are shown in Table 1 below.

TABLE 1

| Parameter | Range |
|---|---|
| Fine particle dose (<5 microns) in mg | 38.82-46.46 mg |
| Delivered dose in mg | 53.2-60.0 mg |
| Respirable fraction (%) | 74.1% |

These results for the DHEAS suspension of the invention can be compared to the saline nebulizer formulation described above in Example 5, and a dry powder inhalation (DPI) formulation. The saline nebulizer formulation had a respirable fraction of 10% and the DPI formulation had a respirable fraction between 30-40%. The suspension formulation of the invention has a respirable fraction of 74.1%, thus demonstrating a striking improvement in respirable fraction over these two formulations.

Breath simulation testing of a clinical batch material of the DHEAS suspension formulation is performed as described above and the results show that 56.5% of the total dose of 70 mg, or 39.41 mg would be delivered in 4.1 minutes. Assuming that 74% of the delivered dose would be respirable, then 29 mg would be produced in the respirable range in 4 minutes. Of this total, some would be lost in anatomic dead space, therefore, the estimated deposited lung dose per vial would be approximately 20 mg. Earlier tests had shown that 10 capsules of a DPI formulation would deliver a maximum of 13 mg to the lung, only under ideal conditions with sufficient inspiratory effort on the patient's part. It would take approximately 10 vials and more than 3 hours to nebulize the saline solution to achieve the same delivered lung dose as one 4 minute nebulization session with the suspension formulation of the invention. Since for most patients, a 3 hour nebulzation would not be practicable, the suspension formulations of the invention provide not just an incremental improvement over prior formulations, but represent a dramatic, enabling improvement over prior DHEAS inhalation formulations.

### Example 7

### In-Vivo Toxicological Assessment

The prototype suspension formulation is tested for toxicologic effects in rats and dogs for 6 weeks. In addition to the chronic toxicology studies, the new suspension formulation is tested for acute effects on the central nervous system, the cardiovascular system, and the respiratory system.

In the dog toxicology study, doses of 0, 5.2, 10.6 and 19.1 mg/kg/day are administered on a daily basis for 6 weeks. Six control animals and four animals in the high dose group are allowed to recover from dosing for an additional 2 weeks. The animals are monitored daily for clinical signs and periodic blood samples are taken to monitor the clinical condition of the animals. At the conclusion of dosing, a complete histopathologic exam is performed on the euthanized animals. The recovery animals are sacrificed two weeks later and examined. There are no toxicopathologic findings attributable to drug at any dose level. The administration of the DHEAS suspension of the invention by inhalation administration for 42 consecutive days at dose levels of saline control, vehicle control, 5.2, 10.6 and 19.1 mg/kg/day is well tolerated and resulted in no adverse reactions to treatment. Thus, the NOAEL (No observable adverse effect level) for this study is 19.1 mg/kg/day, the maximum technically feasible dose.

In the rat toxicology study, doses of 0, 3.47, 7.33 and 16.2 mg/kg/day are administered on a daily basis for 6 weeks. Forty control animals and twenty animals in each of the low and high dose groups are allowed to recover from dosing for an additional 2 weeks. The animals are monitored daily for clinical signs. At the conclusion of dosing, a complete histopathologic exam is performed on the euthanized animals. The recovery animals are sacrificed two weeks later and examined. The administration of the DHEAS suspension of the invention during daily nose-only inhalation administration at dose levels of 3.47, 7.33 and 16.2, in the rat for 6 weeks resulted in transient dose-dependent decreases in food consumption and transient inhibition of bodyweight gain in high dose males exposed to 16.2 mg/kg/day. These findings are no longer present at the end of the recovery period. There are no toxicopathologic findings attributable to drug at any dose level. Thus, the NOAEL (No observable adverse effect level) for this study is 16.2 mg/kg/day, the maximum technically feasible dose.

There are no acute adverse effects noted in the central nervous system, the cardiovascular system or the respiratory system attributable to treatment with the new suspension formulation.

In both species, the pre-dose levels of DHEA-S and DHEA are unmeasurable. After 6 weeks of dosing, there is a several hundred fold increase in DHEA-S over endogenous levels in the dog high dose group but no increase in DHEA. In rats, there is a several thousand fold increase in DHEA-S over endogenous levels in the high dose group and a several hundred fold increase in DHEA over endogenous levels also in the high dose group.

### Example 8

### Comparison of Systemic Exposure Between the Suspension Formulation of the Invention and a Dry Powder Inhalation (DPI) Formulation after Steady State Dosing

The systemic exposure is measured for the rat and dog toxicology studies described above. The data is summarized in Table 2. The delivered dose is much higher for the suspension formulation compared to the dry powder formulation in both rats and dogs. However, in every case, the systemic exposure is less for the suspension formulation than for the dry powder formulation for DHEA-S and for DHEA. These data suggest that the suspension formulation of the invention is more effectively delivered than prior formulations tested. One explanation for these results is that the reduced particle size of the suspension formulation reduces oropharyngeal deposition, thereby reducing systemic absorption and exposure. Similar results are expected to be seen in the human clinical studies.

TABLE 2
Systemic Exposure Data

| | | | | Cmax | | AUC₀₋ₜ | |
|---|---|---|---|---|---|---|---|
| Analyte | Species | Formulation | Highest dose | (ng/mL) | | (ng*hr/mL) | |
| DHEA-S | Rat | DPI | 2.48 mg/kg/day | 2263 | (♂) | 6643 | (♂) |
| | | | | 7218 | (♀) | 23264 | (♀) |
| | | Suspension | 16.2 mg/kg/day | 268 | (♂) | 1018 | (♂) |
| | | | | 968 | (♀) | 4234 | (♀) |
| | Dog | DPI | 3.54 mg/kg/day | 726 | (♂) | 2172 | (♂) |
| | | | | 1007 | (♀) | 2281 | (♀) |
| | | Suspension | 19.1 mg/kg/day | 74 | (♂) | 204 | (♂) |
| | | | | 74 | (♀) | 258 | (♀) |
| DHEA | Rat | DPI | 2.48 mg/kg/day | 46 | (♂) | 185 | (♂) |
| | | | | 107 | (♀) | 444 | (♀) |
| | | Suspension | 16.2 mg/kg/day | 22 | (♂) | 77 | (♂) |
| | | | | 53 | (♀) | 225 | (♀) |
| | Dog | DPI | 3.54 mg/kg/day | 11 | (♂) | 95 | (♂) |
| | | | | 8 | (♀) | 52 | (♀) |
| | | Suspension | 19.1 mg/kg/day | 5 | (♂) | 22 | (♂) |
| | | | | 2 | (♀) | 12 | (♀) |

### Example 9

### Human Clinical Trial Do Demonstrate the Efficacy of the Formulations of the Invention

The primary objective of the clinical study is to determine whether once daily administration of the DHEAS suspension of the invention will improve asthmatic control in patients who remain uncontrolled on low dose inhaled corticosteroid (ICS) and long-acting beta-agonists (LABA).

The secondary objectives of the study are to describe the safety, pharmacokinetics and tolerability of a nebulized formulation of once daily DHEAS suspension in uncontrolled moderate to severe persistent asthmatics on ICS+LABA compared to patients who remain on ICS+LABA and placebo.

The primary endpoint is the change from baseline in the Asthma Control Questionnaire (ACQ) over the 6 week treatment period with an inter-group comparison between the DHEAS suspension of the invention and placebo.

Secondary endpoints is change in morning PEFR, trough FEV1, the Asthma Quality of Life Questionnaire (AQLQ), proportion of withdrawals and changes in hormonal levels and markers of bone turnover for safety. Other exploratory analyses are conducted.

This is a randomized, double blind, parallel group study of once daily dosing of the DHEAS suspension of the invention versus placebo. The run-in phase of the study is characterized by a two-step reduction in ICS dose while maintaining the LABA dose constant. During the run-in period, patients will assess their symptoms and peak flow rates on a daily basis. At the conclusion of the 5-week run-in period, a 24-hour serum profile of endocrine safety parameters and serum profiles of DHEA and DHEAS is obtained from patients. A morning serum cortisol level and 24 hour urinary cortisol is determined as well as serum markers of bone metabolism. ACQ is assessed at every visit. At the conclusion of the Run-In Period, patients must have an FEV1% predicted ≧50 (off beta-agonists) and have an ACQ score of at least 2 for the week prior to randomization in order to be eligible. Eligible patients are randomized to receive either 20 mg (lung dose) DHEAS suspension or placebo once daily using the eFlow nebulizer, in addition to 1 puff twice daily of Seretide# Accuhaler 100/50 (which continues for the duration of the study) for a duration of six weeks.

After randomization, patients return weekly for interim safety and efficacy assessments and for trough in-clinic FEV1 and PEFR determinations and ACQ assessments. During the study, patients monitor their peak flow and symptoms twice daily on an electronic peak flow meter/symptom diary. At Visit 9, AQLQ is administered again. At the end of the Treatment Period, a 24-hour serum profile of endocrine safety parameters, DHEA and DHEAS is obtained from patients. A morning serum cortisol level and 24 hour urinary cortisol is determined as well as serum markers of bone metabolism. The AQLQ is administered at the end of the Treatment Period.

The target patient population are symptomatic moderate to severe persistent asthmatics on either ≧800 μg budesonide+LABA or 1000 μg/day of fluticasone+LABA at a stable dose for at least 3 months prior to screening. Patients may not take any other anti-asthma medication except rescue beta-agonist.

Main Eligibility Criteria:
Moderate to severe persistent asthma patients between the ages of 18 and 65 years of age.
Patients must have a predicted in-clinic FEV1 of ≧60% after withholding bronchodilators (at least six hours for short-acting beta-agonist and 12 hours for long-acting beta-agonist) at screening.
Patients must have <10 pack year smoking history.
Patients must be taking inhaled corticosteroids at doses of at least 800 μg/day budesonide+LABA or at least 1000 μg/day fluticasone+LABA for at least 3 months prior to screening.
Patients may not be on oral glucocorticoids (3-month wash-out), leukotriene receptor antagonists (two week wash-out), systemic anti-IgE therapy (6-month wash-out), calcium supplements, SERMs (Evista etc), bisphosphonates, calcitonin, testosterone replacement therapy or testosterone antagonist therapy.
Patients may continue medications for allergic rhinitis at a constant dose and may continue on immunotherapy.
Patients may take short-acting beta-agonists as needed throughout the study.
Female patients must be willing to use two accepted methods of birth control or be at least one year post-menopausal or surgically sterile. If patients are on oral contraceptives or hormone replacement therapy, they may continue on the therapy at a constant dose throughout the study.

There is a cross group comparison of changes between the DHEAS suspension and placebo for the efficacy and safety endpoints.

The primary comparison is the change in Asthma Control Questionnaire (ACQ) from baseline (defined as the last week prior to randomization during the baseline period) to the ACQ at the end of the treatment period between the DHEAS suspension of the invention and placebo (defined as the last week of the randomized period). The standard deviation of the change from baseline of the ACQ score in the analysis of variance is estimated at 1.0. If the population standard deviation of the change from baseline of the ACQ is 1.0, then 214 randomized subjects are required to achieve 90% power for two-sample t-test to detect a difference of 0.5. A difference of 0.5 units in the ACQ is considered to be clinically relevant.

### ADVANTAGES

The DHEAS suspension of the invention delivered with the eFlow® device has the following benefits which are expected to result in higher efficacy for the following reasons:
The suspension formulation delivers a lung dose between 17-20 mg which is approximately 4 times the minimally effective dose
The eFlow device reliably delivers a high concentration of drug to the lower airways without relying on the patient's inspiratory effort
The smaller particle size of the new suspension formulation will bypass the oropharynx and reduce the systemic absorption and reduce the taste issues
The eFlow device is a battery operated, high efficiency nebulizer that delivers the effective lung dose in 4 minutes
Together, the eFlow device and suspension formulation represent a marked improvement in patient convenience and acceptance over 1) the jet nebulizer/solution formulation combination and 2) the Cyclohaler/DPI combination
Despite the increase in delivered dose, the suspension formulation produced less systemic exposure in dogs and rats in the toxicologic studies

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A composition for inhalation comprising a divalent cation and an aqueous suspension of DHEAS.

2. The composition of claim 1 wherein the divalent ion comprises an alkaline earth metal.

3. The composition of claim 1 wherein the divalent ion comprises magnesium in the form of a water soluble pharmaceutically acceptable salt.

4. The composition of claim 1 wherein the molar ratio of divalent cation to DHEAS is between about 0.5 and 5.

5. The composition of claim 1 wherein the molar ratio of divalent cation to DHEAS is between about 0.25 and 4.

6. The composition of claim 1 wherein the molar ratio of divalent cation to DHEAS is between about 0.75 to 1.25.

7. The composition of claim 1 wherein the amount of DHEAS in the suspension is between about 0.5 wt. % and 10 wt. %.

8. The composition of claim 1 wherein the amount of DHEAS in the suspension is between about 1 wt. % and 10 wt. %.

9. The composition of claim 1 wherein the amount of DHEAS in the suspension is between about 2 wt. % and 5 wt. %.

10. The composition of claim 1 wherein the amount of DHEAS in the suspension is about 3.5 wt. %.

11. The composition of claim 1 further comprising an excipient.

12. The composition of claim 11 wherein the excipient comprises a sugar or sugar alcohol

13. The composition of claim 11 wherein the excipient comprises xylitol or mannitol

14. The composition of claim 1 further comprising a sweetener.

15. The composition of claim 14 wherein the sweetening agent comprises saccharine-sodium or aspartame.

16. The composition of claim 1 further comprising a flavoring agent.

17. The composition of claim 16 wherein the flavoring agent comprises levomenthol.

18. The composition of claim 1 further comprising a preservative.

19. The composition of claim 18 wherein the preservative comprises a methyl, ethyl, or propyl-4-hydroxybenzoate.

20. The composition of claim 1 further comprising an emulsifier or surfactant.

21. The composition of claim 20 wherein the emulsifier or surfactant is Vitamin E-TPGS

22. The composition of claim 1 further comprising a pharmaceutically acceptable buffer for adjusting the pH of the composition to between about 5 and about 8.

23. The composition of claim 22 wherein the pharmaceutically acceptable buffer is for adjusting the pH in a range between about 6 and about 7.5.

24. A composition for inhalation comprising a salt of DHEAS wherein the counterion to DHEA-S comprises a divalent cation.

25. The composition of claim 24 wherein the divalent ion comprises an alkaline earth metal.

26. The composition of claim 24 wherein the divalent ion comprises magnesium.

27. The composition of claim 24 wherein the molar ratio of divalent cation to DHEAS is between about 0.5 and 5.

28. The composition of claim 24 wherein the molar ratio of divalent cation to DHEAS is between about 0.25 and 4.

29. The composition of claim 24 wherein the molar ratio of divalent cation to DHEAS is between about 0.75 to 1.25.

30. The composition of claim 24 further comprising an excipient.

31. The composition of claim 30 wherein the excipient comprises a sugar or sugar alcohol

32. The composition of claim 30 wherein the excipient agent comprises xylitol or mannitol.

33. The composition of claim 24 further comprising a sweetener.

34. The composition of claim 33 wherein the sweetening agent comprises saccharine.

35. The composition of claim 24 further comprising a flavoring agent.

36. The composition of claim 35 wherein the flavoring agent comprises levomenthol

37. The composition of claim 24 further comprising a preservative.

38. The composition of claim 37 wherein the preservative comprises a methyl, ethyl, or propyl-4-hydroxybenzoate.

39. The composition of claim 24 further comprising an emulsifier or surfactant.

40. The composition of claim 39 wherein the emulsifier or surfactant is Vitamin E-TPGS.

41. The composition of claim 24 further comprising a pharmaceutically acceptable buffer for adjusting the pH of the composition to between about 5 and about 8.

42. The composition of claim 41 wherein the pharmaceutically acceptable buffer is for adjusting the pH in a range between about 6 and about 7.5.

43. A method of making or manufacturing an aqueous formulation for nebulization comprising the steps of;
mixing DHEAS in an first aqueous volume;
mixing a compound comprising a divalent cation in a second aqueous volume: and
combining the aqueous volumes to form a suspension of DHEAS.

44. The method of claim 43 further comprising the step of homogenizing the suspension of DHEAS.

45. The method of claim 43 wherein the divalent cation comprises an alkaline earth metal.

46. The method of claim 45 wherein the divalent cation comprises magnesium.

47. The method of claim 43 wherein the compound comprising the divalent cation is magnesium chloride.

48. The method of claim 43 further comprising mixing an excipient into the first aqueous volume, the second aqueous volume, or both the first and second aqueous volumes.

49. The method of claim 48 wherein the excipient comprises xylitol or mannitol.

50. The method of claim 43 further comprising mixing a sweetening agent into the first aqueous volume, the second aqueous volume or both the first and second aqueous volumes.

51. The method of claim 50 wherein the sweetening agent comprises saccharine.

52. The method of claim 43 further comprising mixing a flavoring agent into the first aqueous volume, the second aqueous volume or both the first and second aqueous volumes.

53. The method of claim 52 wherein the flavoring agent comprises levomenthol

54. The method of claim 43 further comprising mixing a preservative into the first aqueous volume, the second aqueous volume or both the first and second aqueous volumes.

55. The method of claim 54, wherein the preservative comprises a methyl, ethyl, or propyl-4-hydroxybenzoate.

56. The method of claim 43 further comprising mixing an emulsifier or surfactant into the first aqueous volume, the second aqueous volume or both the first and second aqueous volumes.

57. The method of claim 56 wherein the emulsifier or surfactant is Vitamin E-TPGS.

58. The method of claim 43 wherein the first aqueous volume is basic.

59. The method of claim 43, further comprising the addition of HCl to the first aqueous volume.

60. The method of claim 43 further comprising homogenizing the suspension formed by mixing the first and second aqueous volumes.

61. A method of making an aqueous composition for inhalation comprising the steps of;
mixing DHEAS sodium salt, an excipient, a preservative, a sweetening agent, an emulsifier and a flavoring agent in an first aqueous volume;
mixing a compound comprising magnesium chloride in a second aqueous volume;
combining the first and second aqueous volumes to form a suspension of DHEAS; and
homogenizing the suspension.

62. The method of claim 61 wherein the excipient comprises xylitol or mannitol.

63. The method of claim 61 wherein the preservative comprises a methyl, ethyl, or propyl-4-hydroxybenzoate.

64. The method of claim 61 wherein the sweetening agent is saccharine.

65. The method of claim 61 wherein the emulsifier is Vitamin E-TPGS.

66. The method of claim 61 wherein the flavoring agent is levomenthol.

67. The method of claim 61 wherein the combining of the first and second aqueous volumes comprises adding the second aqueous to the first aqueous volume in a controlled manner.

68. The aqueous suspension formed from the process comprising the steps of;
mixing DHEAS in a first aqueous volume;
mixing a compound comprising a divalent cation in a second aqueous volume; and
combining the aqueous volumes to form a suspension of DHEAS.

69. The aqueous suspension of claim 68 wherein the divalent ion comprises an alkaline earth metal.

70. The aqueous suspension of claim 68 wherein the divalent ion comprises magnesium.

71. The aqueous suspension of claim 68 wherein the molar ratio of divalent cation to DHEAS is between about 0.5 and 5.

72. The aqueous suspension of claim 68 wherein the molar ratio of divalent cation to DHEAS is between about 0.25 and 4.

73. The aqueous suspension of claim 68 wherein the molar ratio of divalent cation to DHEAS is between about 0.75 to 1.25.

74. The aqueous suspension of claim 68 wherein the amount of DHEAS in the suspension is between about 0.5 wt. % and 20 wt. %.

75. The aqueous suspension of claim 68 wherein the amount of DHEAS in the suspension is between about 1 wt. % and 10 wt. %.

76. The aqueous suspension of claim 68 wherein the amount of DHEAS in the suspension is between about 2 wt. % and 5 wt. %.

77. The aqueous suspension of claim 68 wherein the amount of DHEAS in the suspension is about 3.5 wt. %.

78. The aqueous suspension of claim 68 further comprising an excipient.

79. The aqueous suspension of claim 78 wherein the excipient comprises a sugar or sugar alcohol.

80. The aqueous suspension of claim 79 wherein the excipient agent comprises xylitol or mannitol.

81. The aqueous suspension of claim 68 further comprising a sweetener.

82. The aqueous suspension of claim 81 wherein the sweetening agent comprises saccharine.

83. The aqueous suspension of claim 68 further comprising a flavoring agent.

84. The aqueous suspension of claim 83 wherein the flavoring agent comprises levomenthol

85. The aqueous suspension of claim 68 further comprising a preservative.

86. The aqueous suspension of claim 85 wherein the preservative comprises a methyl, ethyl, or propyl-4-hydroxybenzoate.

87. The aqueous suspension of claim 68 further comprising an emulsifier or surfactant.

88. The aqueous suspension of claim 87 wherein the emulsifier or surfactant is Vitamin E-TPGS.

89. The aqueous suspension of claim 68 further comprising a pharmaceutically acceptable buffer for adjusting the pH of the aqueous suspension to between about 5 and about 8.

90. The aqueous suspension of claim 89 wherein the pharmaceutically acceptable buffer is for adjusting the pH to between about 6 and about 7.5.

91. The aqueous suspension of claim 68 having an osmolality between 200 and 500 mosmol/kg.

92. A method of treating an animal comprising;
nebulizing the composition of claim 1 with a nebulizer capable of an emitted dose of greater than 50% of the nominal dose wherein greater than 50% of the emitted composition comprises droplets less than or equal to about 5 μm in diameter.

93. The method of claim 92 wherein the emitted dose is the dose emitted via mouthpiece or face mask

94. The method of claim 92 wherein the emitted composition has mass median aerodynamic diameter (MMAD) between about 2 to about 5 μm

95. The method of claim 92 wherein the emitted composition has mass median aerodynamic diameter (MMAD) between about 3 to about 4 μm.

96. The method of claim 94 wherein the emitted composition has a geometric standard deviation (GSD) of less than about 2.
